(19) United States
(12) Patent Application Publication
(10) Pub. No.: US 2004/0184949 A1
(43) Pub. Date: Sep. 23, 2004

(54) Air treatment system for localized and personal use

(76) Inventor: John J. McEllen, Chagrin Falls, OH (US)

Correspondence Address: Renner, Kenner, Greive, Bobak, Taylor & Weber, Fourth Floor, First National Tower, Akron, OH, 44308-1456, US

(21) Appl. No.: 10/389,822

(22) Filed: Mar. 17, 2003

(51) Int. Cl. A61L 9/00; A61L 2/00

(52) U.S. Cl. 422/4; 422/121; 422/24

(57) ABSTRACT

In general, the present invention provides a personal air sterilization unit including an intake port; an exhaust port; a sterilization duct communicating in between the intake port and the exhaust port; an ultraviolet light source emitting ultraviolet light and retained within the sterilization duct to expose the interior of the sterilization duct to ultraviolet light; and means for drawing air into the intake port, forcing it through the sterilization duct, where it is treated by ultraviolet light, and thereafter forcing it out through the exhaust port to create a circulating air current encompassing a personal air space.

### BACKGROUND OF THE INVENTION

The present invention generally relates to a self-contained portable air treatment system, particularly for localized and personal use. The air treatment system employs ultraviolet (particularly UV-C) light to disable various airborne microorganisms, including bacteria cells, virus cells, and mold spores and anthrax.

UV-C light sources have been used by hospitals, microbiology labs, food and water and other industries to disable microorganisms that are airborne and/or on work surfaces and equipment and the like. Generally, UV light sources have been incorporated into the irradiation systems and air circulation and ventilation systems to expose harmful microorganisms to UV light in order to reduce the potential for exposure to such microorganisms. Of particular concern herein are those systems of the prior art that are based upon the movement of air past UV sources to disable airborne microorganisms. The devices of the prior art have taken an all-or-nothing approach to the treatment of air within an enclosed area, and it is the focus of the present invention that this all-or-nothing approach is unnecessary, requiring unduly intricate and involved apparatus.

For instance, U.S. Pat. Nos. 3,750,370, 5,523,057 and 6,264,888 deal with physical filtration systems that further employ ultraviolet light to sterilize air passing through the filter elements. These patents, however, focus upon sterilizing an entire room, and generally provide the UV filtration unit in conventional heater and air conditioning system locations. U.S. Pat. Nos. 4,017,736, 5,616,172 and 5,997,619 likewise focus upon sterilizing the entire volume of air within a room, but do so by providing large air purification units that establish an air current that envelops the entire volume of air in a room (or at least a very large portion thereof) in an attempt to purify the entire volume of air in the room. These large units are expensive to manufacture, burdensome to transport to a desired room, and consume an unnecessary amount of energy during operation.

U.S. Pat. No. 6,053,968 provides a portable air purifier that, unlike the large units mentioned above, is lightweight and readily portable. However, the air purifier therein is taught to both intake and exhaust air through an arc measuring substantially 360° with respect to a central axis of an ultraviolet purification section. Thus, the unit therein, while being smaller than other prior art units, still unnecessarily focuses upon purifying the entire contents of a room.

In distinction, the present invention does not have the purification of an entire room as its central focus, although the present invention can be employed for such a purpose. Rather, the present invention advances the art with the realization that what is important is providing sterilized air particularly to the individuals that are present in an enclosed area. Thus, the present invention focuses upon the realization that there is a need in the art for an air purifier for localized and personalized use that ensures a steady supply of sterilized air to the breathing space of an individual in close proximity to the device.

### SUMMARY OF THE INVENTION

In general, the present invention provides a personal air sterilization unit including an intake port; an exhaust port; a sterilization duct communicating between the intake port and the exhaust port; an ultraviolet light source emitting ultraviolet light and retained within the sterilization duct to expose the interior of the sterilization duct to ultraviolet light; and means for drawing air into the intake port, forcing it through the sterilization duct, where it is treated by ultraviolet light, and thereafter forcing it out through the exhaust port to create a circulating air current encompassing a volume of up to about 125 cubic feet, defining a personal air space.

The present invention also provides a method for protecting an individual from exposure to microorganisms and/or molds and for protecting others from exposure to microorganisms and/or molds exhaled by an infected individual. This method includes the steps of circulating air in a targeted partial closed circuit encompassing an individual's personal air space, and treating the air with ultraviolet light to disable microorganisms and/or molds within the air. By “targeted partial closed circuit” it is to be understood that, while the air circulating in the step of circulating air is not absolutely confined to any particular flow pattern, the main distribution of air flow encompasses a somewhat circular or elliptical or conical path that is dependent upon a flow path created between an exhaust port and an intake port. This method may be practiced with the personal air sterilization unit of this invention, which is generally intended to be a small, personal unit that would be placed in close proximity to either a healthy individual wishing to be protected from exposure to microorganisms and/or molds or an infected individual wishing to protect others from exposure to the microorganisms that he may exhale.

In a particular embodiment a method is provided for protecting an individual from exposure to microorganisms and for protecting others from exposure to microorganisms exhaled by an infected individual. The method includes the steps of drawing air through an air sterilization unit having an intake port that spans approximately 180°, an exhaust port that spans approximately 120°, and a sterilization duct communicating between the intake port and exhaust port; and treating the air with an ultraviolet light source within the sterilization duct to disable microorganisms within the air drawn therethrough.

### BRIEF DESCRIPTION OF THE DRAWINGS

For a complete understanding of the objects, techniques and structure of the invention, reference should be made to the following detailed description and accompanying drawings wherein:

FIG. 1 is a front plan view of an air sterilization unit according to this invention, showing the exhaust port in a fully open position;

FIG. 2 is a cross-sectional view of the air sterilization unit of FIG. 1, taken along the line 2-2; and

FIG. 3 is a front plan view of the air sterilization unit of FIG. 1, showing the exhaust port closed to a smaller size, as compared with the exhaust port of FIG. 1

### PREFERRED EMBODIMENT FOR CARRYING OUT THE INVENTION

The device of this invention provides a “personal” air sterilization unit that employs ultraviolet (UV) light to destroy airborne microorganisms and/or molds. The air sterilization unit is particularly configured for personal use by having both a directional intake and directional exhaust. By directing the air that is sterilized by the unit towards an individual's breathing space and directionally intaking exhaled air from that individual's breathing space, the chance of that particular individual becoming infected by another individual in a surrounding environ is significantly reduced. Further, if that individual happens to be infected, the chance for uninfected individuals to become infected by that individual's exhaled germs is significantly reduced, because that individual's exhaled breath is likely to be taken up by the directionally focused intake on the air sterilization unit, more so than by the non-directionally focused intake ports on prior art devices. With this general understanding in mind, a preferred embodiment of the invention is herein disclosed.

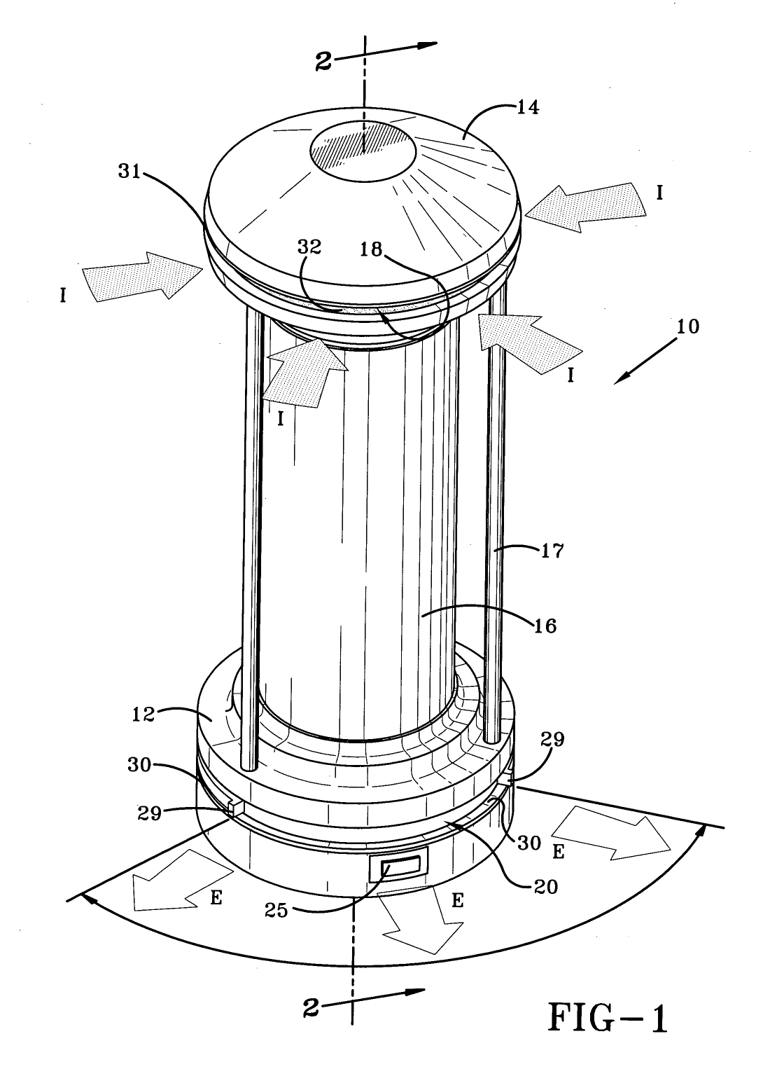

With reference to FIGS. 1-3, it can be seen that a personal air sterilization unit according to this invention is designated generally by the numeral 10. Unit 10, in preferred embodiments, is generally cylindrical, as shown, and includes a base portion 12 and top portion 14, with a sterilization duct 16 communicating therebetween. Each of these elements, and, thus, the general shape of unit 10 need not be cylindrical, as shown, and, indeed, any general shape of unit 10 may be employed and yet fall within the scope of the present invention. Although the present invention is not to be limited to any particular choice of materials, base portion 12 and top portion 14 are preferably constructed steel. Sterilization duct 16 is preferably constructed from brushed aluminum. Tubing 17, which is generally employed for both aesthetics and, in particular embodiments, for providing a wire way for a power supply, as will be described below, communicates between base portion 12 and top portion 14. Tubing 17 may be hollow for provision of a wire way, and is preferably constructed of steel.

The entire unit 10 is intended for personal use and, therefore, is preferably constructed to be both relatively small and lightweight. Unit 10, from the bottom of base portion 12 to the top of top portion 14, preferably ranges from about 14 to about 24 inches in height. The diameters of base portion 12 and top portion 14, which typically will be chosen to be identical, will preferably range from about 5 to about 7 inches. In a particularly preferred embodiment, unit 10 is approximately 17⅜ inches in height and 6 inches wide. Unit 10 will also preferably weigh from about 4 to about 6 lbs., and, in the particularly preferred embodiment, has a net weight of approximately 4 lbs. It should be noted here that the weight of unit 10 takes into account the entirety of unit 10.

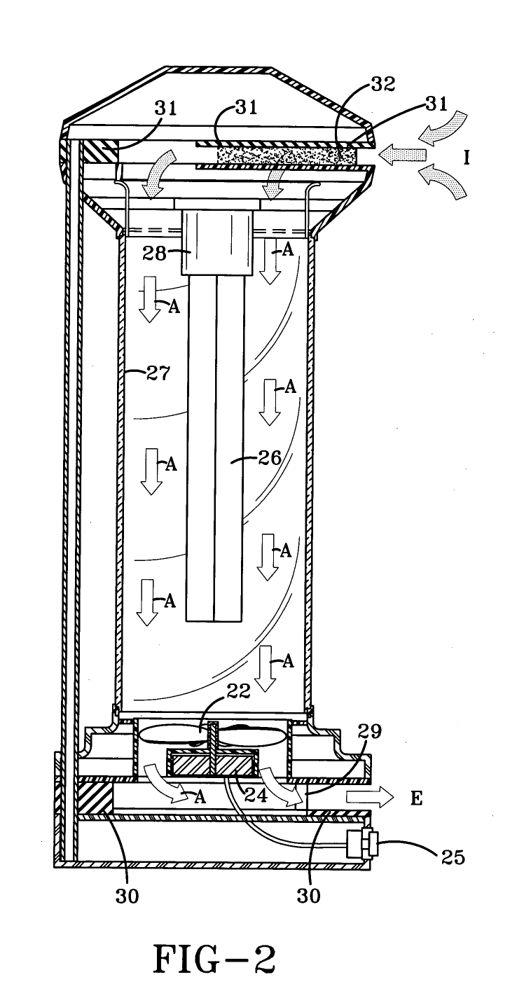

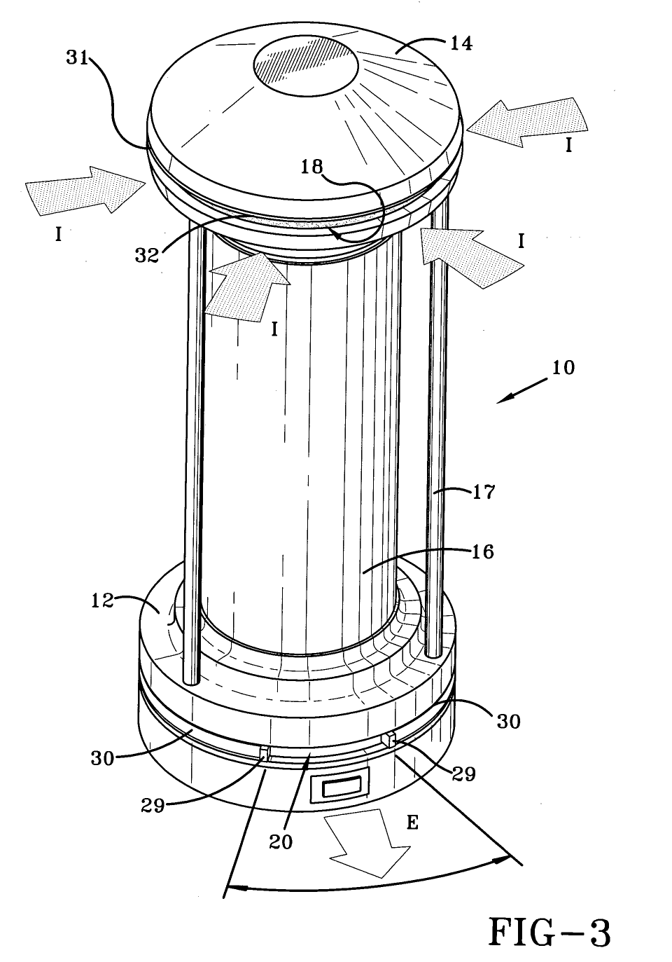

An intake port 18 is provided in top portion 14 and an exhaust port 20 is provided in base portion 12, and each of these ports 18, 20 communicates with the interior of sterilization duct 16 to create an air path, during operation of unit 10, that is generally represented by the arrows designated by the letter A, in the cross-sectional view of FIG. 3. In operation, intake air (represented by arrows I) is directed along air path A by means of fan 22, which is located in base portion 12. Fan 22 is operated by a conventional motor 24, which connects to a suitable power source through conventional methods, and is operable by connection (represented by a non-numbered wire in FIG. 2) to a conventional on/off switch 25. For instance, although not shown in the drawings, an electric cord may connect to motor 24 and run externally of base 12 to be plugged into a suitable power outlet, or, alternatively, motor 24 could be battery operated, and a conventional on/off switch 25 could connect or disconnect the flow of power to motor 24, through known methods.

In operation, fan 22 draws intake air I in at intake port 18 and draws the air downward, through sterilization duct 16, as represented by air path A, and out exhaust port 20, as exhaust air E. At least one ultraviolet light source 26 (UV source 26) is disposed within sterilization duct 16, such that air taken in at intake port 18 and forced through the interior of sterilization duct 16 is treated with ultraviolet light before being forced out of unit 10 at exhaust port 20. The interior wall 27 of sterilization duct 16 is preferably reflective to increase the UV dosage at the interior wall 27. In preferred embodiments, interior wall 27 is coated with materials such as brushed, polished, or sputtered aluminum, and the like, in order to provide a reflective interior surface.

UV source 26 is operated by means of an appropriate power source 28, which, like motor 24, may take many conventional forms. Power source 28 is, for the most part, simply generically represented in FIG. 2, it being understood that the provision of power to ultraviolet light source 26 will be handled in a conventional manner. Both motor 24 and power source 28 may communicate with individual power switches or with a single power switch, as shown at numeral 25, and already discussed above in FIGS. 1 and 2. With this single power switch 25 in base 12, tubing 17 serves as a wire way for the supply of power to UV source 26. In a conventional manner, an electric power cord may communicate with power switch 25 in order to provide power to both fan 22 and UV source 26 upon the operation of switch 25. Likewise, batteries could be employed as the ultimate power source.

Bacillus anthracis, e-coli, strep, staph, rhinovirus (common cold), influenza, small pox, measles, legionella, ebola, tularemia and other microorganisms are susceptible to UV-C. To a lesser degree, molds (eg., airborne spores from aspergillum niger—“black mold,” and anthrax spores) are also susceptible to UV-C. Notably, UV-A and UV-B are universally present in sunlight, and it is widely held that bacteria and viruses have not and cannot mutate to resist susceptibility to UV light. Advantageously, UV-C, while being present in sunlight, is blocked by the ozone layer, and, consequently, microorganisms are generally not exposed to UV-C, and, thus, are extremely vulnerable to this form of UV light, which destroys they by altering their DNA. UV-C is a more aggressive and effective UV light. A precise dosage of UV-C has been proscribed to irradiate various microorganisms, since each is susceptible to UV-C in varying degrees. For over 50 years, dosage requirements to disable over 90% of various microorganisms have been characterized by the exposure to UV-C in joules per square meter (J/m²). Unit 10 is preferably designed to produce a minimum exposure to airborne microorganisms of from about 30 to about 75 J/m² on the outer perimeter of the inside portion of the cylinder, while the air flow is set on a fixed level.

Fan 22 is preferably designed to draw and force air along air path A at a rate of about 5 to about 12 cubic feet per minute (cfm). In the particularly preferred embodiment of this invention, a 25 cfm fan is employed, and a flow of about 8.5 cfm is realized along air path A due to the fact that the flow is contained by protective measures and baffling to prevent “leaking” of UV-C from the unit.

Although other measures might be taken, in this embodiment, UV-absorbing adjustable spacer 30 and UV-absorbing spacer 31 are respectively provided in bottom portion 12 and top portion 14. Particularly, spacers 30, 31 help define exhaust port 20 and intake port 18. Light is scattered by the reflectance of the inside surface of sterilization duct 16, and absorbed by spacers 30, 31, which are to be constructed from UV-absorbing materials, such as glass or polycarbonate or acrylic. Adjustable spacer 31 is adjustable at tabs 29 to define the directional limits of exhaust port 20. Spacer 30, in the preferred embodiment herein is not adjustable, but rather is shaped substantially like a washer that is made thinner at intake port 18 to define the same. Adjustable spacer 31 is also shaped substantially like a washer, with the adjustable portion being the thick portion of the washer that is movable at tabs 29 to adjust the directional limits (degree of arc) of exhaust port 20.

It is the focus of the present invention to provide a sterilized personal air space such that the sterilization unit 10 of this invention will be particularly useful for localized and personalized use ensuring a steady supply of sterilized air to the breathing space of an individual in close proximity to the device. Thus, the present invention provides both intake port 18 and exhaust port 20 with directional capabilities. Particularly, intake port 18 is configured to span through an maximum of approximately 180° with respect to the central axis of sterilization duct 16. Exhaust port 20 is configured to be adjustable to exhaust air through an arc measuring from about 60° to about 120° with respect to this central axis of sterilization duct 16, as represented between the two FIGS. 1 and 3. Thus, unit 10 is most effective at sterilizing air in a space residing at a particular position with respect to unit 10, namely, in the general direction in which intake port 18 and exhaust port 20 point. Notably, the center lines of the arcs defined by intake port 18 and exhaust port 20 are preferably aligned with one another. With such a design, unit 10 is a preventative device that reduces individual exposure to infection by airborne microorganisms and/or molds by directing a flow of sterilized air toward a target (approximately the breathing space of an individual in close proximity to unit 10). Due to the inertia of the intake and exhaust currents that are created exteriorly of unit 10, by fan 22, and competition with air currents in the immediate environs, unit 10 may be effective at providing a “closed circuit” of sterilized air at varying distances from unit 10. Moreover, unit 10 has been designed to cause a portion of the exhausted air to be overtaken by the intake of the device, at a distance ranging from about 1.5 to about 6 feet, depending upon the power of fan 22. Thus, the “closed circuit” previously mentioned is created by constantly recirculating an amount of air already sterilized, and the benefits to the user are increased and help to counteract exposure to non-sterilized air.

The focus of this invention is to provide an air sterilization unit for personal and localized use that can prevent an individual from exposure to bacteria, viruses, and mold spores, and that can protect others in a room from exposure to an infected individual's exhaled (or otherwise airborne) germs. Thus, the closed circuit of sterilized air preferably provides a circulating air current encompassing a volume of up to about 125 cubic feet, which volume is considered herein to define a practical upper limit for what may be considered a “personal airspace.” An air current encompassing a volume of 125 ft³ would provide a pocket of sterilized air that is approximately 5 feet wide by 5 feet deep by 5 feet high. This volume serves as a desired upper limit, and it will be appreciated that smaller volumes of circulating air currents can be achieved and practiced with beneficial results. In a particularly preferred embodiment, a circulating air current encompassing 60 ft³ is achieved through airflow (fan speed) and venting designed to enclose an individual in a pocket of air that is 5 feet wide by 4 feet deep by 3 feet high.

As mentioned, unit 10 recirculates sterilized air because intake port 18 is designed to overtake sterilized air exhausted at exhaust port 20. This allows the user to realize the benefits of sterilized air more quickly than in devices of the prior art, which do not teach overtaking sterilized air in this manner, and which focus upon circulating air currents that encompass entire rooms (i.e., much greater than the targeted <125 ft³ personal airspace that is the focus of this invention). In the preferred embodiment treating 60 ft³, intake part 18 overtakes the exhausted sterilized air at a distance of about 6 feet, and benefits are realized within approximately 5 minutes of operation. However, this is not to insinuate that an entire room cannot be treated with unit 10. Indeed, due to the fact that natural air currents within a room move air throughout the room, unit 10 will, after a sufficient duration of operation, sterilize an entire practically sized room.

Unit 10 is thus also intended to function as an added protection in the event of biological attack. For instance, in light of the potential for biological terrorist attacks, both in the United States and elsewhere, it is recommended that preparations be made for the sealing of a small area in the home with plastic sheeting and duct tape, and, in the event of a biological attack, a sealed 10×10×8 foot room could be sterilized by the preferred unit 10 in approximately 90 minutes. Unit 10 can serve to protect occupants from any penetration by germs/microorganisms, and, in the event of more than one individual taking shelter, would protect each occupant from the microorganisms that might be introduced by the other occupant.

To avoid introducing certain larger particulates into sterilization duct 16, such as pollen and dust of approximately 0.03 microns or larger, an open cell (eg. HEPA) or other suitable filter 32 is preferably placed interiorly of intake port 18. Thus, along air path A, air may be filtered by a primary filter 32 and, thereafter, continue into sterilization duct 16, where UV source 26 bombards the air with ultraviolet radiation to kill microorganisms and/or molds that may have passed through the optional primary filter 32.

UV source 26 is preferably a source of short wave UV light. This light is also known as “UV-C” light and corresponds to a wave length of light of 253.7 nm. Particularly preferred UV-C light sources are also “ozone free,” being opaque at 185 nm. Ozone generation can occur as a result of the exposure of oxygen to light at 185 nm. Therefore, the glass (i.e. bulb portion) of UV source is preferably treated to suppress all such energy, so that ozone is not generated. It is understood that prolonged exposure to short wave UV light should be avoided and, thus, unit 10 is also constructed so as to protect the user from over exposure to UV irradiation. Particularly, unit 10 is designed to restrict UV-C emmissions to minuscule levels, i.e., less than about 0.6 microwatts per cm² at approximately 6 inches from the area of greatest potential for exposure, which is generally the exhaust port. The National Institute of Occupational Safety and Health (NIOSH) standards recommend that this level of UV-C irradiation is the maximum exposure rate over an 8 hour period. This is accomplished by the spacers 30, 31 as already discussed above. Additionally, it is preferred that steps be taken to prevent access to UV source 26 when UV source 26 is emitting UV light. This may be accomplished by having mating electrical elements between top portion 14 and UV source 26 connected in such a way that the electrical elements must be disconnected before top portion 14 can be fully removed to allow access to UV source 26.

In light of the foregoing, it should thus be evident that the process of the present invention, providing an air treatment system for localized and personal use, substantially improves the art. While, in accordance with the patent statutes, only the preferred embodiments of the present invention have been described in detail hereinabove, the present invention is not to be limited thereto or thereby. Rather, the scope of the invention shall include all modifications and variations that fall within the scope of the attached claims.

What is claimed is:

1. A personal air sterilization unit comprising:
an intake port;
an exhaust port;
a sterilization duct communicating between said intake port and said exhaust port;
a UV light source emitting UV light and retained within said sterilization duct to expose the interior of said sterilization duct to UV light;
means for drawing air into said intake port, forcing it through said sterilization duct, where it is treated by the UV light, and thereafter forcing it out through said exhaust port to create a circulating air current encompassing a volume of up to about 125 cubic feet, defining a personal airspace.

2. The personal air sterilization unit according to claim 1, wherein said exhaust port is adjustable and directs air forced out thereof in an arc selectively measuring from about 60 to about 120 degrees with respect to a central axis of said sterilization duct.

3. The personal air sterilization unit according to claim 2, wherein said intake port transfers air into said sterilization duct through an arc measuring about 180 degrees with respect to said central axis of said sterilization duct.

4. The personal air sterilization unit according to claim 3, wherein sterilized air is exhausted from said adjustable exhaust port and a portion of the exhausted sterilized air is overtaken by the intake port, to create a partial closed circuit of sterilized air within said personal airspace.

5. A method for protecting an individual from exposure to microorganisms and for protecting others from exposure to microorganisms exhaled by an infected individual comprising the steps of:
circulating air in a targeted partial closed circuit encompassing an individual's personal airspace; and
treating the air with an ultraviolet light source to disable microorganisms within the air.

6. A method for protecting an individual from exposure to microorganisms and for protecting others from exposure to microorganisms exhaled by and infected individual comprising the steps of:
drawing air through a air sterilization unit having an intake port that spans approximately 180°, an exhaust port that spans approximately 120°, and a sterilization duct communicating between the intake port and exhaust port; and
treating the air with an ultraviolet light source within the sterilization duct to disable microorganisms within the air drawn therethrough.
